(19) United States
(12) Patent Application Publication
(10) Pub. No.: US 2002/0076408 A1
(43) Pub. Date: Jun. 20, 2002

(54) Combination radiation therapy and chemotherapy in conjunction with administration of growth factor receptor antibody

(76) Inventor: Donald J. Buchsbaum, Birmingham, AL (US)

Correspondence Address: Hendricks and Associates, P. O. Box 2509, Fairfax, VA, 22031-2509, US

(21) Appl. No.: 10/004,833

(22) Filed: Dec. 7, 2001

Related U.S. Application Data
(60) Provisional application No. 60/251,787, filed on Dec. 8, 2000.

(51) Int. Cl. A61K 39/395; A61K 31/704; A61K 33/24; A61K 31/4745; A61K 31/337

(52) U.S. Cl. 424/143.1; 514/34; 424/649; 514/50; 514/449; 514/283

(57) ABSTRACT

This invention comprises a method of inhibiting tumor growth in tumors having growth factor receptors comprising administering, about simultaneously, antibodies to the target growth factor receptors, at least one chemotherapeutic agent and radiation therapy.

This application takes priority from Provisional Patent Application No. 60/251,787 filed Dec. 8, 2001.

### FIELD OF THE INVENTION

This invention is related to use of combination chemotherapy and radiation therapy in conjunction with administration of antibody to growth factor receptors such as epidermal growth factor receptor and Her-2/neu receptor.

### BACKGROUND OF THE INVENTION

Growth factors and their receptors play a key role in the development and progression of human cancers. Epidermal growth factor receptor (EGFR) is a 170-kD transmembrane glycoprotein composed of an extracellular ligand-binding domain, a transmembrane region, and a cytoplasmic protein kinase domain involved in signaling pathways essential for cell division and tumor growth. Transforming growth factor-alpha (TGF-a), EGF and similar ligands bind to the extracellular domain of EGFR and activates its intracellular tyrosine kinase domain. Cells that secrete TGF-a can stimulate their own growth by activating their EGFR. Binding of specific ligands such as epidermal growth factor (EGF) and TGF-a to the extracellular domain results in EGFR dimerization and autophosphorylation, activation of the receptor's cytoplasmic tyrosine kinase domain, and initiation of multiple signal transduction pathways that regulate tumor cell growth and survival. EGFR expression is increased in many types of epithelial tumors and this typically correlates with aggressive tumor growth, as well as with poor clinical outcome. For example, EGFR is overexpressed on 22-60% of human pancreatic carcinomas. A correlation was found between the co-expression of EGFR, EGF or TGF-a, and survival in pancreatic cancer. These studies suggest that EGFR may be a target for therapy of pancreatic cancer.

Monoclonal antibodies (mAb) directed against EGFR that competitively block ligand-binding sites and inhibit ligand-mediated tyrosine kinase phosphorylation have been generated and shown to inhibit TGF-a and EGF-mediated proliferation of EGFR-rich cells both in vitro and in human tumor xenografts in athymic nude mice. IMC-C225 (ERBITUX™) is a human-mouse chimerized IgGl antibody with high affinity to the EGFR, derived from the murine anti-EGFR mAb 225 developed and characterized. The chimeric anti-EGFR mAb IMC-C225 blocks binding of the natural ligands EGF and TGF-a to EGFR and inhibits EGF/TGF-a induced activation of this receptor tyrosine kinase. IMC-C225 has been shown to be cytotoxic and inhibit the proliferation of tumor cells in vitro and the growth of tumor xenografts in athymic nude mice

In both in vitro and in vivo pre-clinical studies, mAb 225 or IMC-C225 was shown to enhance the anti-tumor effects of the chemotherapeutic agents doxorubicin, cisplatin, paclitaxel, and topotecan. Cisplatin+IMC-C225 produced responses in patients with head and neck cancer

Previous studies have shown that systemically administered unlabeled monoclonal antibodies that bind to growth factor receptors expressed on tumor cells (e.g. epidermal growth factor receptor and Her-2/neu receptor) facilitate tumor growth inhibition and, when combined with chemotherapy or radiation therapy (RT), result in enhanced tumor growth inhibition. However, there has been no previous suggestion that antibodies that bind to growth factor receptors be used in conjunction with both chemotherapy and radiation therapy simultaneously.

### SUMMARY OF THE INVENTION

It is the purpose of this invention to provide improved means for the treatment of malignancies wherein growth factor receptors appear to play a role in tumor growth. Broadly, the invention comprises a method of inhibiting tumor growth in tumors having growth factor receptors comprising administering, about simultaneously, antibodies to the target growth factor receptors, at least one chemotherapeutic agent and radiation therapy.

It has now been demonstrated that the systemic administration of such antibodies that bind growth factor receptors in conjunction with simultaneous administration of both radiation therapy (RT) and chemotherapy will provide improved cure rates. The data provided herein shows the value of combination therapy using growth factor receptor antibody, chemotherapy and radiation therapy against tumors that are ordinarily treated with radiation, such as head and neck cancer, renal cell cancer, pancreatic cancer, colon cancer, lung cancer, brain cancer, etc.

DETAILED DESCRIPTION OF THE INVENTION

In accord with the purpose of the invention to provide improved means of treatment using antibodies to growth factors in conjunction with chemotherapy and irradiation, two antibodies which have been designated IMC-C225 and Herceptin were evaluated in use (1) alone, (2) in conjunction with chemotherapy or (3) in conjunction with irradiation and (4) antibody in conjunction with both chemotherapy and irradiation treatment. The methods of the invention were exemplified and evaluated using pancreatic cancer cells in vitro and pancreatic cell implants in mice.

Materials and Methods:

The antibody IMC-C225 was obtained from ImClone Systems, Inc, of New York, N.Y. 10014. Human pancreatic cancer cell lines, BxPC-3 and MiaPaCa-2 and human colon cancer cell line SW948 were obtained from the American Type Culture Collection (ATCC, Manassas, Va.). A431 human epidermoid cancer cells were also obtained from the ATCC.

Early stages of apoptosis were identified using an annexin V-FITC apoptosis detection kit (BioVision Research Products, Palo Alto, Calif.). In studies of radiation cell survival assays, survival data was fitted to the linear quadratic (LQ) and single hit multi-target (SHMT) radiobiological models using Fit v. 2.4 software kindly provided by Dr. N. Albright, University of California at San Francisco.

Various combinations of treatment modality were studied in vitro. IMC-C225, a chimeric monoclonal antibody to epidermal growth factor receptor (EGFr), inhibits tumor cell proliferation and enhances cytotoxicity of drugs or radiation. In an evaluation of interaction of IMC-C225 combined with gemcitabine (GEM) plus radiation therapy (RT) on EGFr moderately expressing Mia-PaCa-2 human pancreatic cancer cells it was found IMC-C225+GEM+RT and GEM+RT were equivalent and produced greater inhibition of tumor cell proliferation as well as apoptosis in vitro compared to IMC-C225 (5 μg/ml), RT (3 Gy), GEM (27 nM), IMC-C225+RT, or IMC-C225+-GEM. Similar clonogenic survival occurred following IMC-C225 (5 μg/ml)+RT(8 Gy) or IMC-C225+GEM (50 nM)+RT.

Studies were then done in vivo to evaluate the same active agents alone or in combination. Athymic nude mice were implanted subcutaneously with 2×10⁷ Mia-PaCa-2 cells mixed with Matrigel. Twenty-two days later, mice were injected intraperitoneally with IMC-C225 (1 mg q3d×12), GEM (120 mg/kg q6d, starting 1 day after the first dose of IMC-C225), and RT to the tumor (3 Gy q6d, beginning at 1 day after GEM). Combined treatment of mice bearing tumors (n=8/group) showed enhancement of complete tumor regressions following treatment with IMC-C225+GEM+RT (8/8) compared to IMC-C225+RT (5/8), IMC-C225+GEM (0/8), GEM+RT (1/8), IMC-C225 (1/8), GEM (0/8), or RT (0/8). Animals treated with IMC-C225, GEM, RT, IMC-C225+GEM, IMC-C225+RT, GEM+RT, or IMC-C225+GEM+RT had mean tumor size doubling times of 46, 22, 35, 59, 201, 101, and 309 days, respectively. Surprisingly, the in vivo data demonstrated greatly improved results when IMC-C225, GEM, and RT were used in combination, a finding contra to the findings on in vitro studies disclosed above.

In Vitro Cell Proliferation:

Even though IMC-C225 blocked EGF-activated tyrosine kinase activity in pancreatic cancer cell lines, the ability of IMC-C225 to inhibit cell proliferation in vitro was different between the two cell lines. After exposure for 96 h to IMC-C225, the BxPC-3 cells showed a 35% reduction in cell proliferation as compared to untreated cells. Under the same conditions, MiaPaCa-2 cells did not show any inhibitory effect by IMC-C225. A 24 h exposure to the IC₅₀ dose of gemcitabine inhibited approximately 55% of the BxPC-3 cell growth whereas approximately 45% inhibition was observed with the MiaPaCa-2 cells. When IMC-C225 was included 24 h prior to the addition of gemcitabine, a modest decrease in cell proliferation was observed in BxPC-3 cells when compared to gemcitabine treatment alone. MiaPaca-2 cells were not affected by the addition of IMC-C225 24 h prior to gemcitabine treatment compared to gemcitabine treatment alone.

Radiation treatment (3 Gy) was given on day 2 either alone or in combination with IMC-C225 and/or gemcitabine treatment. When radiation was combined with IMC-C225, there was a decrease in cell proliferation compared to radiation or IMC-C225 treatment alone for the BxPC-3 cells, but not for the MiaPaCa-2 cells. The combination of radiation and gemcitabine produced a decrease in cell proliferation both for BxPC-3 and MiaPaCa-2 cells and the addition of IMC-C225 treatment did not enhance this effect.

Apoptotic Cell Death

The induction of apoptosis was determined using an identical treatment schedule for cell cycle and cell proliferation assays. For BxPC-3 cells, there was not a significant increase in apoptosis among the IMC-C225+gemcitabine group, the gemcitabine+radiation group and the three-agent combination of IMC-C225+gemcitabine+radiation group. However these three groups produced a significant increase in apoptotic cell death as compared to all other treatment groups (p-value<0.05).

For MiaPaCa-2 cells, the gemcitabine treatment resulted in a small but significant increase in apoptotic cell death which was not enhanced by any other treatment (p-value<0.05).

Animal Model Tumor Therapy Studies

Female BALB/c athymic nude mice, 4-6 weeks old, were purchased from the National Cancer Institute Frederick Cancer Research and Development Center (Frederick, Md.). The mice were used when they were 8-9 weeks old. Human pancreatic tumor cells were suspended in serum-free growth medium. Cell viability was determined by trypan blue dye exclusion. BxPC-3 cells were mixed (1:1) with Matrigel (Collaborative Biomedical Products, Bedford, Mass.) and 2×10⁷ cells injected subcutaneously (s.c.) into athymic nude mice on day 0. Following tumor injection, mice were returned to their sterile micro-isolator cages and maintained on autoclaved lab-chow and sterile water, ad libitum.

Animal experiments included seven treatment groups: IMC-C225 alone, gemcitabine alone, radiation alone, IMC-C225+gemcitabine, IMC-C225+radiation, gemcitabine+radiation, and IMC-C225+gemcitabine+radiation. Radiation treatment was delivered via a ⁶⁰Co therapy unit (Picker) using a custom-designed mouse holder that exposed the tumor bearing dorsal flank for irradiation while shielding normal tissues. Beginning on day 15 after BxPC-3 tumor cell injection, four groups of 8 mice each were injected intraperitoneally (i.p.) with 1 mg IMC-C225 every 3 days for 6 weeks total treatment. Four groups of mice received gemcitabine (120 mg/kg) i.v. on days 16, 22, 28, 34, 40, and 47. In four groups of mice, tumors were exposed to six fractions of 3 Gy ⁶⁰Co radiation on days 17, 23, 29, 35, 41, and 48. This was based on reports that gemcitabine 24 h before radiation produced greatest radiosensitization. Serial measurements of s.c. tumor size over time were taken. Tumor size was measured in two dimensions using Vernier calipers three times per week, and the change in tumor surface area (product of the two diameters) compared to the baseline tumor size on the first day of antibody injection (day 15) was determined. Percent change from baseline was computed by comparing the baseline value to the tumor size on each day of measurement. The experiment involving BxPC-3 tumors was terminated after 161 days.

MiaPaCa-2 cells were mixed with Matrigel (1:1) and 2×10⁷ cells injected s.c. into athymic nude mice on day 0. Beginning on day 22 after tumor cell injection, four groups of 8 mice each were injected i.p. with 1 mg IMC-C225 every 3 days for 6 weeks total treatment. Four groups of mice received gemcitabine (120 mg/kg) i.v. on days 23, 29, 35, 41, 47, and 54. In four groups of mice, tumors were exposed to six fractions of 3 Gy ⁶⁰Co radiation on days 24, 30, 36, 42, 48, and 55. The results are presented as the change in tumor size relative to baseline size on day 22. The MiaPaCa-2 experiment was terminated after 333 days.

To determine equivalency in tumor sizes among mice within and between treatment groups at baseline, the tumor size data at baseline were analyzed. For each experiment, a one-way ANOVA was used to test for differences in the mean tumor size between the 7 treatments. The associated ANOVA yielded no statistical differences between any of the treatment groups (p-value=0.683). The test for homogeneity of variances was not significant (p-value=0.568), i.e., variances between treatments were not significantly different from each other, and the error terms did not significantly deviate from a normal distribution. Thus, it was concluded that the data do not demonstrate differences with respect to mean tumor size or variability in tumor size amongst the treatment groups at the start of treatment. Similarly, the ANOVA for the BxPC-3 pancreatic tumor study yielded no significant differences (p-value=0.121) and the test for homogeneity of variances was not significant (p-value=0.399). As a confirmatory measure, the analysis was repeated using the natural-log transformation on size confirming the results on the raw data, i.e., similar significant differences between groups in the same ranking with very similar p-values, and residual analysis revealing no serious violations in model assumptions.

In the MiaPaCa-2 experiment, there were a total of 2 mice that were sacrificed due to sickness, 41 sacrificed due to tumor size (over four times original size), 4 actual “natural” deaths and a total of 9 mice survived till study termination at day 333. Interestingly, 6 of the 9 animals that survived over the entire time course were within one group, the triple therapy combination (IMC-C225+gemcitabine+3 Gy) group. Fisher's exact test comparing proportions of surviving mice between groups yielded a highly significant result (p-value<0.001). Thus, the IMC-C225+gemcitabine+3 Gy treatment group had a significantly larger number of tumors that had delayed growth. Note that only 1 animal in this group was sacrificed due to tumor size.

In the MiaPaCa-2 study, 14 of the tumors underwent complete regression, with 3 recurring before study ending date. For those tumors that regressed, the average number of days to recurrence was approximately 60 with a standard deviation of 25.8 (most of these survived till the study ending at day 333 with the average survival approximately 292 days with a standard deviation of 63.8. The 14 tumor regressions occurred in only three specific groups, 5 (62.5%) in the IMC-C225+3 Gy group, 1 (12.5%) in the gemcitabine+3 Gy group and 8 (100%) in the IMC-C225+gemcitabine+3 Gy group, with zero regressions occurring in the other four treatment groups. The overall Fisher's exact test indicated that the observed dependence of regression on treatment was highly significant (p-value<0.001). The comparison of regression occurrence between gemcitabine+3 Gy and those groups with zero regressions was not significant (p-value=0.20), between IMC-C225+gemcitabine+3 Gy and the zero regression groups was highly significant (p-value<0.001), and the test comparing IMC-C225+gemcitabine+3 Gy with gemcitabine+3 Gy was not significant (p-value=0.056).

In the BxPC-3 experiment, there were a total 2 mice that were sacrificed due to sickness, 46 sacrificed due to tumor size (over four times original size), 2 sacrificed due to tumor ulceration, and only 6 mice survived till study termination. The p-value resulting from the Fisher's exact test of this data was not significant (p-value=0.191).

In the BxPC-3 study, almost all tumors in surviving mice eventually doubled from their original size before termination of the study. The triple treatment group had an average time to tumor doubling greater than all other treatment groups. An ANOVA was conducted to determine if the observed mean differences were significant. The resulting p-value<0.01 indicates that these differences were indeed significant.

Immunochemistry

MiaPaCa-2 and BxPC-3 xenograft bearing animals received one week of treatment with IMC-C225 (two injections of 1 mg at a 3 day interval), gemcitabine (120 mg/kg, 1 day after the first dose of mAb), and radiation (3 Gy at 1 day after gemcitabine). This one week treatment regimen was used to reduce the amount of necrosis that would occur with a full course of therapy. At 4 days after radiation, animals were injected i.p. with BrdU and killed 2 h later. The tumors were excised, fixed in alcoholic formalin (Pen-Fix) and stained for EGFR, BrdU, and apoptosis. Our methods for performing and evaluating immunohistochemistry have been reported in the literature. Interference from endogenous mouse antibodies was reduced by high temperature citric acid antigen retrieval and the use of HistoMouse BEAT Blocker (Zymed Laboratories, Inc., San Francisco, Calif.). The anti-BrdU antibody (DAKO Corp., Carpinteria, Calif.) was used at a 1:40 dilution. After removing paraffin and rehydration, and antigen retrieval, the tissue sections were incubated with 3.5 N HCl for 15 min to denature the DNA followed by immunohistochemistry.

Apoptosis was evaluated in tissue using the TUNEL technigue (Apoptag Kit, Intergen, Purchase, N.Y.) to detect the free 3′-OH ends of double or single stranded DNA via the enzymation incorporation of digoxigenin, which is recognized by an antibody coupled to horseradish peroxidase following a reaction with DAB. Light hematoxylin was used for counter-staining. After staining, apoptosis was determined as the proportion of cells with apoptotic nuclei from 1,000 randomly selected cells.

The results showed that treatment produced an increase in apoptosis in MiaPaCa-2 cells (30-40%) compared to the basal level in untreated tumors (5-10%), while there was a marked reduction in proliferation detected by BrdU staining in treated MiaPaCa-2 tumors (10-15%) compared to the basal level in untreated tumors (70-80%). In contrast, BxPC-3 treated tumors showed no increase in apoptosis (0.5%) or proliferation (15-20%) compared to untreated tumors. Extensive individual cellular death was observed and EGFR expression was reduced in treated MiaPaCa-2 tumors compared to untreated tumors, whereas no increase in individual cell death and no change in EGFR expression was noted in BxPC-3 tumors. These immunohistochemistry results directly correlate with the differences observed in tumor growth following treatment for these two pancreatic tumors

Statistical Analyses

In the animal model tumor therapy studies, multiple endpoints were examined and appropriate statistical tests were conducted to address differences between treatment groups with respect to each endpoint. The endpoints included, the size of the tumor, which was computed for each tumor at each time period, percent of original tumor size, computed for each tumor at each time point after baseline, regression, time to regression, and time that the tumor size reached twice the original tumor size (time to double). Inferences are made on the true median rather than the mean response, i.e. median tumor size or median time to double in size.

Clearly, the combination of IMC-C225, GEM and RT has application in the treatment of malignancies wherein the EGFr is a factor in proliferation of tumor cells.

While the chemotherapeutic agent exemplified herein was gemcitabine, other agents may be used. The choice of agent will be determined in view of the tumor type and the clinical evaluation of the physician. For example, the following chemotherapeutic agents are examples:

head and neck cancer: cisplatin, 5-fluorouracil

colon cancer: 5-fluorouracil, irinotecan (CPT-11), cisplatin, paclitaxel

pancreatic cancer: gemcitabine, 5-fluorouracil, irinotecan

brain cancer: gemcitabine, 5-fluorouracil

lung cancer: irinotecan

prostate: doxorubicin, cisplatin

Other malignancies wherein EGFr is expressed include breast (often treated with doxorubicin or paclitaxel) and ovarian (often treated using cisplatin or paclitaxel) malignancies. The dosage for use with any therapeutic agent in the combination therapy would usually be on the order of that generally administered when the agent is used alone.

It would also be appropriate to use antibodies against growth factor receptors such as Her-2/neu or epidermal growth factor receptor in combination with both radiation therapy and two or more chemotherapy agents. The dosage range for administration of antibodies to EGFr would be about 100 to 3000 mg/kg. At the present time, in adults receiving radiation treatment, an initial dosage of 400 mg/kg IMC-C225 is administered on day one with 250 mg/kg being administered weekly thereafter. For administration in conjunction with both chemotherapy and radiation therapy, the dosage would be continued during the entire period of therapy. This often translates into 6-8 weeks of administration during the usual term of treatment with radiation.

It is surprising that benefit seen in vivo when IMC-C225+GEM+RT are administered simultaneously in comparison with GEM+RT was not demonstrated in in vitro. It was only in the intact animal that the benefits of the treatment methods taught herein were demonstrated.

Studies Using Herceptin:

Studies in accord with the studies described above using Herceptin (obtained and available from the pharmacy, University of Alabama at Birmingham Medical Center) in place of IMC-C225 were conducted. Athymic nude mice were implanted s.c. with 2×10⁷ MAI PaCa-2 cells mixed with Matrigel. Nineteen days later, mice were injected i.p. with Herceptin anti-erbB-2 monclonal antibody (available commercially from Genentech and presently used for treatment of breast cancer), 1 mg every 3 days for 12 injections, gemcitabine, 6 doses of 120 mg/kg every 6 days, starting day 1 after the first dose of Herceptin, and radiation to the tumor, 6 doses of 3 Gy every 6 days at 1 day after gemicitabine. In this instance, 15/64 of the tumors underwent complete regression, with 14 recurring before the study ending date. For those tumors that regressed, the average number of days to reoccurrence was approximately 47. The 15 tumor regressions occurred in four specific groups, 4 (50%) in the Herceptin+3 Gy group, 2 (25%) in the gemcitabine+3 Gy group, 1 (12.5%) in the Herceptin group, and 8 (100%) in the Herceptin+gemcitabine+Gy group, with no regressions occurring in the other three treatment groups. The Herceptin+gemcitabine+3 Gy group had substantially larger numbers of complete regressions than all other treatment groups.

Studies Using CPT-11 in Colon Cancer”

Studies were also done in vivo to evaluate IMC-C225+CPT-11+RT against human colon tumors. Athymic nude mice were implanted subcutaneously with 2×10⁷ SW948 cells Twenty-two days later, mice were injected intraperitoneally (i.p.) with IMC-C225 (1 mg q3d×12), CPT-11 (33 mg/kg intravenously q4d, starting day 1 after the first dose of IMC-C225), and RT to the tumor (3 Gy q6d, beginning at 1 hour after CPT-11). Combined treatment of mice bearing tumors (n-7/group) showed enhancement of complete tumor regressions following treatment with IMC-C225+CPT-11+RT (3/7) compared to IMC-C225+RT (1/7) or CPT-11 (0/7). Animals treated with CPT-11, IMC-C225+RT or IMC-C225+CPT-11+RT had mean tumor size doubling times of 57, 76 and >98 days, respectively.

While IMC-C225, available from ImClone Systems, Inc., and Herceptin, available from Genentech, have been used to exemplify the invention, other antibodies to growth factor receptors such as EGFr or Her-2/neu receptor could be used in the method of the invention.

While the use of antibodies with either chemotherapy or irradiation have been known, it is probable that the use of the antibodies with chemotherapy and irradiation as a preferred method of treatment have not previously been discovered because the incremental value of the use of the three agents is not seen in in vitro studies.

What we claim is:

1. A method of inhibiting tumor growth in tumors having growth factor receptors comprising administering, about simultaneously, antibodies to the target growth factor receptors, at least one chemotherapeutic agent and radiation therapy.

2. The method of claim 1 wherein the first dose of antibodies to target growth factor receptors is administered before or at the time of administration of at least one chemotherapeutic agent.

3. The method of claim 1 wherein the antibody is to a epidermal growth factor receptor or a Her-2/neu receptor.

4. The method of claim 1 wherein the chemotherapeutic agent is chosen from among cisplatin, irinotecan (CPT-11), paclitaxel, gemcitabine, 5-fluorouracil, and doxorubicin.

5. The method of claim 1 wherein the tumor growth to be inhibited is a pancreatic tumor.

6. The method of claim 1 wherein the tumor growth to be inhibited is a colon tumor.

7. The method of claim 1 wherein the antibody administered is one chosen from IMC-C225 and Herceptin.

8. The method of claim 7 wherein the antibody administered is Herceptin.

9. The method of claim 2 wherein the antibody administered is one chosen from IMC-C225 and Herceptin.

10. The method of claim 4 wherein the chemotherapeutic agent is gemcitabine.

11. The method of claim 1 wherein the antibodies administered are antibodies against epidermal growth factor receptor.

12. The method of claim 1 wherein the course of treatment is at least 6 weeks.

13. The method of claim 12 wherein the antibodies against a growth factor receptor are administered at a higher dosage at the first dose than at subsequent doses.
